(19) United States
(12) Patent Application Publication
(10) Pub. No.: US 2013/0261552 A1
(43) Pub. Date: Oct. 3, 2013

(54) DRUG DELIVERY DEVICE WITH HOUSING COMPRISING FRANGIBLE ZONE

(71) Applicants: Michael Jugl, Frankfurt am Main (DE); Axel Teucher, Frankfurt am Main (DE)

(72) Inventors: Michael Jugl, Frankfurt am Main (DE); Axel Teucher, Frankfurt am Main (DE)

(73) Assignee: SANOFI-AVENTIS DEUTSCHLAND GMBH, Frankfurt am Main (DE)

(21) Appl. No.: 13/992,330

(22) PCT Filed: Dec. 20, 2011
(86) PCT No.: PCT/EP2011/073381

§ 371 Date: Jun. 7, 2013

(30) Foreign Application Priority Data
Dec. 21, 2010 (EP) 10196225.6

(51) Int. Cl. A61M 5/50

(52) U.S. Cl. CPC A61M 5/50
USPC 604/110

(57) ABSTRACT

The present invention relates to a drug delivery device for injecting a dose of a medicament, comprising of a cartridge holder adapted to house a cartridge filled with the medicament and comprising a displaceable piston, and at least a body adapted to house a drive mechanism comprising a piston rod to be operably engaged with the piston of the cartridge for expelling a dose of the medicament, wherein the cartridge holder and the body are interconnected with each other and wherein the cartridge holder and/or the body comprise at least one fractionizing means adapted to irreversibly abrogate the interconnection of cartridge holder and body.

### CROSS REFERENCE TO RELATED APPLICATIONS

The present application is a U.S. National Phase Application pursuant to 35 U.S.C. §371 of International Application No. PCT/EP2011/073381 filed Dec. 20, 2011, which claims priority to European Patent Application No. 10196225.6 filed Dec. 21, 2010. The entire disclosure contents of these applications are herewith incorporated by reference into the present application.

### FIELD OF THE DISCLOSURE

The present invention relates to a drive mechanism for a drug delivery device that allows a user to select single or multiple doses of an injectable medicament and to dispense the set dosage of the medicament as well as to apply said medicament to a patient, preferably by injection. In particular, the present invention relates to such devices, which are handled by the patients themselves.

### BACKGROUND

Drug delivery devices allowing for multiple dosing of a required dosage of a liquid medicinal product, such as liquid medicaments, and further providing administering of the liquid to a patient, are as such well-known in the art.

Drug delivery devices of this kind have to meet a number of user specific requirements. For instance in case of those with diabetes, many users will be physically infirm and may also have impaired vision. Therefore, these devices need to be robust in construction, yet easy to use, both in terms of the manipulation of the parts and understanding by a user of its operation. Further, the dose setting must be easy and unambiguous and where the device is to be disposable rather than reusable, the device should be inexpensive to manufacture and easy to dispose. In order to meet these requirements, the number of parts and steps required to assemble the device and an overall number of material types the device is made from have to be kept to a minimum.

Typically, the medicament to be administered is provided in a cartridge having a displaceable piston or bung mechanically interacting with a piston rod of a drive mechanism of the drug delivery device. By way of the piston rod, thrust can be applied to the piston in distal direction and a certain amount of the medicinal fluid can be expelled from the cartridge.

Drug delivery devices, such like pen-type injectors further comprise multiple housing components, for instance a cartridge holder adapted to receive a cartridge filled with the medicament as well as a pen body housing or body adapted to receive and to house the drive mechanism which is to be operably engaged with the piston of the cartridge. In particular with disposable pen-type injectors, the entire drug delivery device is intended to be discarded after consumption or after use of the medicament stored in its cartridge.

Since the cartridge is typically made of glass or comparable material being inert to the medicament disposed therein, the cartridge and the housing and/or the functional components of the drug delivery device should be discarded or recycled in separate ways. Proper recycling or discarding of the drug delivery device therefore requires separation of the cartridge from the drug delivery device, which by virtue of its disposable design is not possible, because the drug delivery device is generally not intended to be disassembled.

It is therefore an object of the present invention to provide a drug delivery device of disposable type which provides an effective means to disassemble or to fractionize at least the housing components of the drug delivery device in order to enable separate recycling of the cartridge and the device components. It is a further object to provide a respective method for fractionizing or for decomposing the disposable drug delivery device in a well-defined and controlled way. Furthermore, it is intended to implement and/or to separate cartridge and device components in a cost-saving and efficient way, e.g. by only introducing minor amendments to the design of existing drug delivery devices.

### SUMMARY

The present invention provides a drug delivery device for injecting a dose of a medicament. The device comprises at least two housing components, for instance a cartridge holder and a body. The cartridge holder is adapted to house and to receive a cartridge filled with the medicament to be dispensed. The cartridge, typically designed as vial, carpule or ampoule comprises a barrel, typically made of glass or of comparable inert material which is sealed by way of a displaceable piston.

By exerting pressure to the piston, e.g. in distal direction, hence towards a patient, a respective pressure builds up inside the cartridge, thereby urging a well-defined dose of the liquid medicament through a dispensing outlet of the cartridge which is typically in fluid-communication with a piercing element, like an injection needle or cannula to be removably mounted on a distal end section of the cartridge holder. The body of the drug delivery device is typically adapted to house a drive mechanism comprising a piston rod or a drive ram to be operably engaged with the piston of the cartridge for exerting distally directed pressure to the cartridge for expelling a dose of the medicament.

The drug delivery device is preferably designed as a disposable device. Hence, cartridge holder and body are interconnected with each other in such a way, that a repeated disassembly and re-assembly is not possible. Therefore, if the medicament stored in the cartridge is used up or when the drug delivery device is only intended for non-regular but only temporary use, the entire device is intended to be discarded. Since most of the components of the housing and/or the drive mechanism comprise thermoplastic material or metal, a material separation, especially a separation of cartridge and device components should be provided in an easy and intuitive way.

For this purpose, the cartridge holder and/or the body comprise at least one fractionizing means that is adapted to irreversibly abrogate the interconnection of cartridge holder and body. By applying or activating the fractionizing means, cartridge holder and body are irreversibly disconnected from each other and may be separated accordingly. In this released and/or separated configuration, the cartridge disposed inside the cartridge holder can be removed from the cartridge holder and can be discarded or recycled in a separate way. Since the fractionizing means is adapted to irreversibly abrogate the interconnection of cartridge holder and body, misuse or operating errors, e.g. a user trying to replace an empty cartridge with a disposable drug delivery device, can be effectively prevented. The irreversible disassembly of the housing components, cartridge holder and body by means of the fractionizing means therefore enhances patient safety.

In a first preferred embodiment, the fractionizing means comprises at least one bendable and/or pivot-mounted lug disposed at the cartridge holder and/or at the body. Preferably, bending and/or pivoting of the lug is accompanied by a plastic deformation of said lug. This way, the lug cannot return in its interlock configuration and cartridge holder and body cannot re-connect, once the lug has pivoted into its release configuration.

In another preferred aspect, the fractionizing means is integrally formed with the body and/or with the cartridge holder. Preferably, body and/or cartridge holder comprise a thermoplastic component, e.g. manufactured by injection moulding. By integrally forming the bendable or pivot-mounted lug with the body and/or with the cartridge holder, a separate assembly of the fractionizing means with the cartridge holder and/or body is not required. This way, implementation of the fractionizing means into the drug delivery device can be attained in a cost efficient way.

Preferably, the mutual interaction of cartridge holder, body and fractionizing means can be designed such, that a release configuration of cartridge holder and body can only be attained, if the bendable and/or pivot-mounted lug has been displaced in such a way, that a plastic deformation of the lug takes place, thus effectively preventing an elastic return into its initial interlocking configuration.

According to another preferred embodiment, body and/or cartridge holder mutually overlap in an interface section when mutually interconnected. In said interface section, the body comprises a receptacle portion which is adapted to receive a corresponding insert portion of the cartridge holder. However, a diametrically opposite design is also conceivable, wherein a receptacle is provided at a proximal end of the cartridge holder while a distal end section of the pen body housing comprises an insert portion to be positioned therein.

In this way, cartridge holder and body can be arranged in an intertwined or interleaved manner providing a rather rigid and reliable mutual fastening of cartridge holder and body.

In still another aspect, the body and the cartridge holder are positively engaged by means of the fractionizing means. For instance, the fractionizing means may provide a snap-in or clipping feature by way of which the body and the cartridge holder remain interconnected as long as the lugs of the fractionizing means remain inactive. As soon as the lugs of the fractionizing means are activated, e.g. by bending or pivoting, said positive engagement of body and cartridge holder is abrogated, such that body and cartridge holder can be separated from each other in a non-returning way.

Furthermore and according to another preferred aspect, the at least one lug is disposed on the receptacle portion of the body or cartridge holder and comprises a radially inwardly extending protrusion which is adapted to engage with a corresponding receptacle disposed on the insert portion of cartridge holder or body. Depending on the design of the interface of cartridge holder and body, the lug may be disposed on the outer circumference of the receptacle of the body or cartridge holder while the corresponding receptacle is disposed on the outside of the respective insert portion, either of cartridge holder or body.

In order to emphasize and to facilitate mechanical manipulation of the fractionizing means, the lug, in another aspect comprises a structurally weakened section defining a pivot- or bending axis. By providing a groove at a bottom section of the lug, a kind of predetermined bending point or axis for bending and/or pivoting the lug can be provided.

It is of the further benefit, when the pivot- or bending axis defined by the structurally weakening extends substantially parallel or perpendicular to the longitudinal axis of the body or the cartridge holder. When body or cartridge holder comprise a substantially cylindrical geometry, the pivot- or bending axis for the fractionizing means preferably extends parallel to the longitudinal axis of the body or of the cartridge holder, hence in axial direction. However, if the pivot- or bending axis extends for instance perpendicular to the longitudinal axis of body or cartridge holder, the lug may be arranged in a rather flattened surface section of the receptacle portion in order to enable a smooth executable bending or pivoting of the lug.

In still another preferred embodiment, the lug, at least when in its initial interlocking configuration, flushes with the outer circumference of the receptacle portion of body or cartridge holder. This way, unintentional displacement of the lug can almost be prevented since manipulation of the lug requires a rather sophisticated lifting of the lug's free end.

Therefore, and according to another preferred embodiment, the receptacle portion of either body or cartridge holder comprises a slit opposite a free end section of the lug. Said slit comprises a slit width or a size that allows to lift the free end of the lug, e.g. by the help of a fingernail or by means of a tool of comparable size.

In still another preferred aspect, the receptacle portion of body or cartridge holder is provided with an adhesive cover or foil covering the fractionizing means and its lug or lugs. By adhering a protective element across the at least one lug, unintentional activation, in particular a bending or pivoting of said lug can be prevented. For fractionizing and disassembling the drug delivery device it is first required to remove the adhesive cover in order to get access to the fractionizing means.

In another embodiment, the lug itself, preferably its free end section may be separately attached and connected to the protective foil. It may even be permanently connected to the lug. This way, a pivoting or bending of said lug into its release configuration can be attained while removing the protective foil from the respective housing component, cartridge holder or body. Hence, a manual handling and lifting of the free end section of the lug may become superfluous.

In still another aspect, the drug delivery device is readily equipped with a cartridge positioned and fixed by the cartridge holder, wherein the cartridge is filled with the medicament to be dispensed. Moreover, the drive mechanism is already operably engaged with the piston of the cartridge when the drug delivery device is delivered to the end-customer.

Finally, the various components of the drug delivery device, in particular its cartridge and its housing or the functional components of its drive mechanism are intended to be separately discarded after consumption or use of the medicament.

By making use of the fractionizing means, the drug delivery device can be disassembled and fractionized, such that at least the cartridge, typically comprising a glass barrel can be removed from the cartridge holder and can be discarded or recycled separately.

In still another and independent aspect, the invention further relates to a method of fractionizing a drug delivery device after its use, wherein the drug delivery device comprises at least a body and a cartridge holder that are interconnected in an interface section in a mutually interleaved manner. In said interface section, a receptacle portion of body or cartridge holder receives an insert portion of the cartridge holder or body, respectively. In particular the method is applicable to a drug delivery device as described above.

The method of fractionizing the drug delivery device comprises the steps of irreversibly pivoting or bending a fractionizing means, arranged at the outer circumference of the receptacle portion of cartridge holder or body, into a release configuration, in which cartridge holder and body are mutually released. In the next step, body and cartridge holder are separated from each other in order to gain access to the cartridge disposed in the cartridge holder. Thereafter, the cartridge is removed from the cartridge holder and is discarded or recycled separately from the housing or functional components of the drug delivery device.

This way, even a disposable drug delivery device, such like a pen-type injector intended to be discarded after usage can become subject to an environmentally friendly discarding- or recycling process.

The term, medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(w-carboxyheptadecanoyl)-des(B30) human insulin and B29-N-(w-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(0)14 Trp(O2)25, Asp28] Exendin-4(1-39),

### des Pro36 [Met(0)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(0)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;
or an Exendin-4 derivative of the sequence

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(0)14, Asp28] Exendin-4(1-39)-Lys6-NH2,

### des Met(0)14 Asp28 Pro36, Pro37, Pro38 Exendin-4(1-39)-NH2,

H-(Lys)6-desPro36, Pro37, Pro38 [Met(0)14, Asp28] Exendin-4(1-39)-NH2,

### H-Asn-(Glu)5-des Pro36, Pro37, Pro38 [Met(0)14, Asp28] Exendin-4(1-39)-NH2,

### des Pro36, Pro37, Pro38 [Met(0)14, Asp28] Exendin-4(1-39)-(Lys)6-NH2,

### H-(Lys)6-des Pro36, Pro37, Pro38 [Met(0)14, Asp28] Exendin-4(1-39)-(Lys)6-NH2,

### H-Asn-(Glu)5 des Pro36, Pro37, Pro38 [Met(0)14, Asp28] Exendin-4(1-39)-(Lys)6-NH2,

### H-Lys6-des Pro36 [Met(0)14, Trp(O2)25, Asp28] Exendin-4(1-39)-Lys6-NH2,

### H-des Asp28 Pro36, Pro37, Pro38 [Met(0)14, Trp(O2)25] Exendin-4(1-39)-NH2,

### H-(Lys)6-des Pro36, Pro37, Pro38 [Met(0)14, Asp28] Exendin-4(1-39)-NH2,

### H-Asn-(Glu)5-des Pro36, Pro37, Pro38 [Met(0)14, Trp(O2)25, Asp28] Exendin-4(1-39)-NH2,

### des Pro36, Pro37, Pro38 [Met(0)14, Trp(O2)25, Asp28] Exendin-4(1-39)-(Lys)6-NH2,

### H-(Lys)6-des Pro36, Pro37, Pro38 [Met(0)14, Trp(O2)25, Asp28] Exendin-4(S1-39)-(Lys)6-NH2,

### H-Asn-(Glu)5-des Pro36, Pro37, Pro38 [Met(0)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the pertinent art that various modifications and variations can be made to the present invention without departing from the spirit and scope of the invention. Further, it is to be noted, that any reference signs used in the appended claims are not to be construed as limiting the scope of the present invention.

### BRIEF DESCRIPTION OF THE DRAWINGS

In the following, preferred embodiments of the invention will be described in greater detail by making reference to the drawings in which:

FIG. 1 exemplary illustrates a drug delivery device of pen-type injector,

FIG. 2 shows an enlarged view of the interface of cartridge holder and pen body housing,

FIG. 3 schematically illustrates a cross section along A-A- according to FIG. 2 with the fractionizing means in interlock configuration and

FIG. 4 shows the cross section according to FIG. 3 with fractionizing means in release configuration.

### DETAILED DESCRIPTION

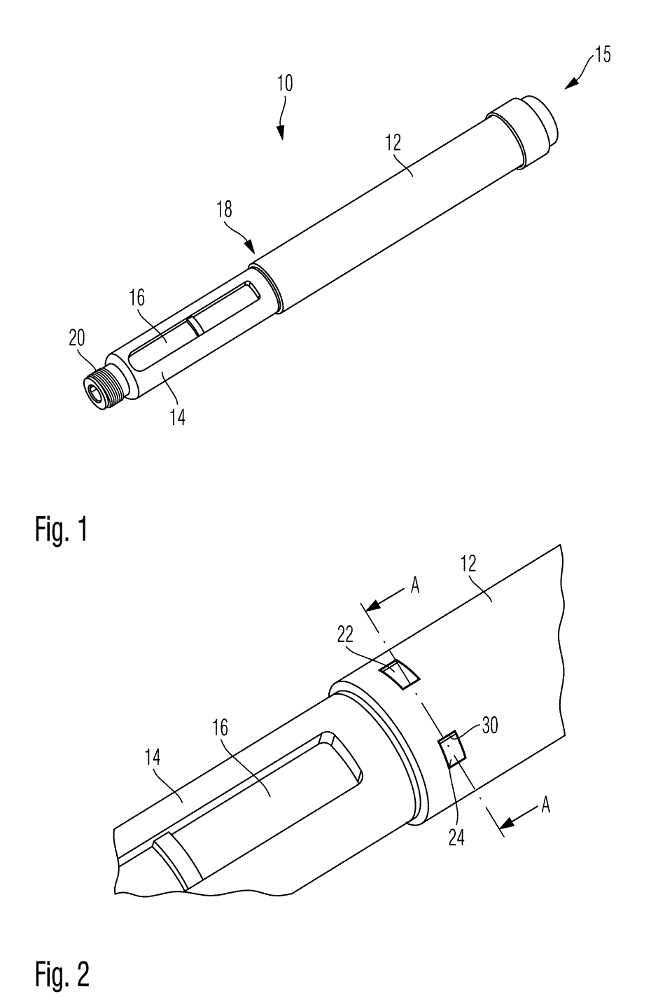

The drug delivery device 10 as depicted in FIG. 1 comprises a pen body housing 12 connected with a cartridge holder section 14, in which a cartridge 16 is disposed. In the illustrated sketch, the cartridge 16 is only visible through an inspection window provided in the cartridge holder 14. The cartridge holder 14 at its distal end section comprises a threaded socket 20 adapted to receive a correspondingly threaded needle assembly having an injection needle intended to pierce a distally located sealing member of the cartridge 16, which is typically designed as a septum.

Opposite its distal outlet, the cartridge 16 comprises a displaceable piston to operably engage with the piston rod or drive ram of a drive mechanism that is housed in the body 12. Body 12 and cartridge holder 14 are interconnected by forming an interface 18 in an interleaved and mutually overlapping manner. In the illustrated embodiment, the distal end of the body 12 comprises a receptacle adapted to receive a proximally located insert portion of the cartridge holder 14. Furthermore, at a proximal end of the body 12, a dose button 15 is located allowing to manipulate and to control dose setting and dose dispensing of the drug delivery device 10.

The illustrated drug delivery device 10 is preferably of disposable type. Hence, when the drug delivery device is not intended for regular but only temporary use, the entire device 10 should be discarded when a treatment with the medicament has terminated. Otherwise, for patients regularly using the pen-type injector 10, the entire device 10 is to be discarded when the medicament provided in the cartridge 16 is used up.

In order to enable separate recycling and environmentally friendly separate discarding of cartridge 16 and the residual components of the drug delivery device 12, 14, 15 a fractionizing means 22, 24 is provided at the outer circumference of the distal portion of the body 12 that overlaps in radial direction with a proximal insert portion of the cartridge holder 14.

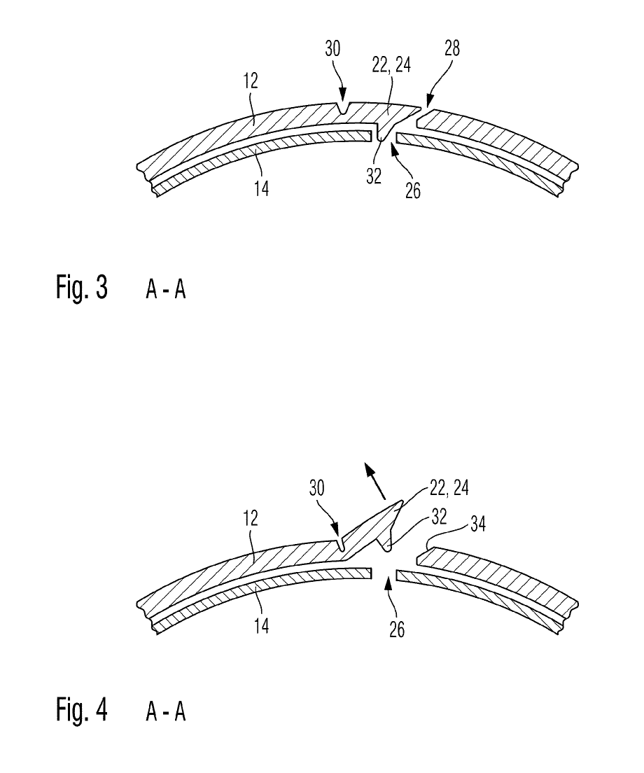

In the illustrated embodiment, the fractionizing means 22, 24 comprises pivot-mounted or bendable lugs or flaps 22, 24 that allow and enable irreversible disassembly of cartridge holder 14 and body 12. As illustrated in the cross sections according to FIGS. 3 and 4, the bendable lugs 22, 24 are integrally formed with the pen body housing 12. Further, a groove 30 arranged at a socket portion of the lug 22, 24 serves as a predetermined bending or pivoting axis, which in the present embodiment extends substantially parallel to the longitudinal axis of cartridge holder 14 or pen body housing 12. Since lugs 22 and body 12 are integrally formed, the groove 30 provides a structurally weakened portion of the housing, thereby defining the bending or pivot axis.

The lugs 22, 24 in their initial configuration substantially flush with the outer circumference of the body 12. However, the free end section of the lugs 22, 24 correspond with an inclined and bevelled surface portion 34 of a circumferentially adjacent body portion. This way, in the initial configuration according to FIG. 3, a slit 28 is formed allowing to insert a fingernail or a tool of corresponding size for gripping and/or for lifting up of the clip-like fractionizing means 22, 24.

The pivot-mounted or bendable lug 22, 24 comprises a radially inwardly protruding nose or protrusion 32 adapted to engage with a receptacle or with a through opening 26 disposed in the insert portion of the cartridge holder 14. As long as a mutually engaging configuration as illustrated in FIG. 3 is maintained, cartridge holder 12 and body 14 remain positively engaged and mutually interlocked.

As soon as the flap-like lug 22, 24 is lifted into its release configuration as depicted in FIG. 4, mutual engagement of body 12 and cartridge 14 is abrogated, the positive interlock is abolished and cartridge holder 14 and body 12 can be separated from each other, e.g. along the longitudinal direction. The shape and geometry as well as the material of the lug 22, 24 and its weakening groove 30 is selected such, that a release configuration as depicted in FIG. 4 is only attainable when the lug is plastically deformed. This way, a returning of the lug 22, 24 in its interconnecting configuration as depicted in FIG. 3 is not possible.

Even though FIG. 2 only depicts two bendable lugs 22, 24, the interface region of body 12 and cartridge holder 14 may be equipped with numerous lugs, e.g. equidistantly surrounding the outer circumference of the receptacle portion of cartridge holder 14 or body 12, respectively.

1-9. (canceled)

10. Drug delivery device for injecting a dose of a medicament, comprising:
a cartridge holder housing a cartridge filled with the medicament and comprising a displaceable piston,
a body housing a drive mechanism comprising a piston rod to be operably engaged with the piston of the cartridge for expelling a dose of the medicament,
wherein the cartridge holder and the body are interconnected with each other and
wherein the cartridge holder and/or the body comprise at least one fractionizing means adapted to irreversibly abrogate the interconnection of cartridge holder and body to remove the cartridge from the cartridge holder, characterized in that
the fractionizing means comprise at least one bendable and/or pivot-mounted lug disposed at the cartridge holder and/or at the body, wherein the body and/or the cartridge holder mutually overlap in an interface section, in which the body comprises a receptacle portion which is adapted to receive an insert portion of the cartridge holder, or vice versa, and wherein the lug is disposed on the receptacle portion of the body or cartridge holder and comprises a radially inwardly extending protrusion adapted to engage with a corresponding receptacle disposed on the insert portion of cartridge holder or body.

11. The drug delivery device according to claim 10, wherein the fractionizing means is integrally formed with the body and/or with the cartridge holder.

12. The drug delivery device according to claim 10, wherein the body and the cartridge holder are positively engaged by means of the fractionizing means.

13. The drug delivery device according to claim 10, wherein the lug comprises a structurally weakened section defining a pivot- or bending axis.

14. The drug delivery device according to claim 13, wherein the pivot- or bending axis extends substantially parallel or perpendicular to the longitudinal axis of the body or cartridge holder.

15. The drug delivery device according to claim 10, wherein the lug flushes with the outer circumference of the receptacle portion of body or cartridge holder when in interlock configuration.

16. The drug delivery device according to claim 10, wherein the receptacle portion comprises a slit opposite a free end section of the lug.

17. The drug delivery device according to claim 10, wherein the receptacle portion of body or cartridge holder is provided with an adhesive cover or foil covering the fractionizing means.

18. A method of fractionizing a drug delivery device according to claim 10 after its use, the method of fractionizing the drug delivery device comprises the steps of:
irreversibly pivoting or bending a fractionizing means disposed at the outer circumference of the receptacle portion into a release configuration,
separating body and cartridge holder in order to gain access to the cartridge disposed therein,
removing the cartridge from the cartridge holder and discarding the cartridge separate from the housing components of the drug delivery device.
